(19) United States
(12) Patent Application Publication
(10) Pub. No.: US 2009/0143433 A1
(43) Pub. Date: Jun. 4, 2009

(54) COCKTAIL FOR MODULATION OF ALZHEIMER'S DISEASE

(76) Inventor: Curt Hendrix, West Lake Village, CA (US)

Correspondence Address: VENABLE LLP, P.O. BOX 34385, WASHINGTON, DC, 20043-9998, US

(21) Appl. No.: 12/325,842

(22) Filed: Dec. 1, 2008

Related U.S. Application Data
(63) Continuation-in-part of application No. 12/149,075, filed on Apr. 25, 2008, now abandoned.
Application No. 12/149,075 is a continuation of application No. 11/293,425, filed on Dec. 1, 2005, now abandoned.
Application No. 11/293,425 is a continuation-in-part of application No. 11/002,750, filed on Dec. 1, 2004, now abandoned.
Application No. 11/002,750 is a continuation-in-part of application No. 11/116,997, filed on Apr. 27, 2005, now abandoned.
Application No. 11/116,997 is a continuation-in-part of application No. 11/002,750, filed on Dec. 1, 2004, now abandoned.
(60) Provisional application No. 60/632,681, filed on Dec. 1, 2004.
(60) Provisional application No. 60/996,702, filed on Nov. 30, 2007.

(51) Int. Cl. A61K 31/4525; A61P 25/28

(52) U.S. Cl. 514/321

(57) ABSTRACT

Formulations for the prevention and treatment of neurological diseases and cognitive deficiencies, i.e., Alzheimer's Disease (AD), Parkinson's Disease, amyotrophic lateral sclerosis, mild cognitive impairment and other types of dementia, comprise therapeutically effective amounts of curcumin, piperine, epigallocatechin-3-gallate (EGCG) and n-acetylcysteine. The combination addresses some or all of the pathways which can result in neurological deficiencies, degeneration and diseases.

This application is a Continuation-in-Part of U.S. application Ser. No. 12/149,075, filed Apr. 25, 2008, which is a Continuation of U.S. application Ser. No. 11/293,425, filed Dec. 1, 2005, which is a Continuation-in-Part of U.S. application Ser. No. 11/002,750, filed Dec. 1, 2004 and U.S. application Ser. No. 11/116,997, filed Apr. 27, 2005, and which claims benefit of U.S. Provisional Appl. No. 60/632,681, filed Dec. 1, 2004. This application also claims the benefit of U.S. Provisional Appl. No. 60/996,702, filed Nov. 30, 2007. Each of these prior applications is incorporated by reference.

This invention has been developed pursuant to NIH grant number 1R43AT003025-01. The Government may have rights in this invention.

### FIELD OF THE INVENTION

This application is directed to new formulations for the prevention and treatment of neurological diseases and cognitive deficiencies, i.e., Alzheimer's Disease (AD), Parkinson's Disease, amyotrophic lateral sclerosis, mild cognitive impairment and other types of dementia. The formulations comprise therapeutically effective amounts of curcumin, piperine, epigallocatechin-3-gallate (EGCG) and n-acetylcysteine. The combination addresses some or all of the pathways which can result in neurological deficiencies, degeneration and diseases.

### BACKGROUND

Alzheimer's disease (AD) is the leading cause of dementia in the elderly. It is generally characterized by a loss of cognitive abilities, including memory, and a rapid deterioration in personality and the ability to care for oneself. Over 5 million Americans are currently diagnosed with AD, and this number could triple over the coming decades as the population ages. One in 10 people aged 65 and over, and around 1 in 2 over the age of 85, develop the disease. Researchers have generally found that the disease itself manifests with the appearance of several hallmark pathologies, including the accumulation of amyloid β (Aβ) peptide and hyperphosphorylated tau proteins. Large inflammatory responses are also seen, along with evidence of oxidative damage. Extensive synaptic and neuronal loss is also frequently observed in AD patients.

AD is a staggeringly expensive disease, costing the American economy more than $83.9 billion annually. The Alzheimer's Association estimates that by 2050 between 11.3 million and 16 million Americans will be AD victims and that the overall economic impact of the disease will increase four-fold. Much of the cost of AD is borne by Medicare and Medicaid. There is currently no treatment that halts the overall progression of the disease.

The standard of care for patients with AD is treatment with anticholinesterase inhibitors. Cholinesterase inhibitors increase the synaptic availability of the neurotransmitter acetylcholine by preventing it from breaking down. Anticholinesterase inhibitors act to slow progression of the disease (particularly deterioration in cognitive function and overall functioning) and often delay the need for institutionalization by several months. Unfortunately, the effect of anticholinesterase inhibitors is only temporary. No treatment currently exists that prevents, halts, or reverses the neurodegenerative process.

A successful treatment for AD will have to address both the accumulation of aggregating biomolecules, such as Aβ and hyperphosphorylated tau, as well as the loss of synapses and neurons. One promising approach is to prevent the development of pathologies in the first place, which is likely to at least delay the onset of the disease. Such a treatment should be safe for prolonged use, and well tolerated by the general population.

### SUMMARY

There is a great need for a significant breakthrough in Alzheimer's prevention and treatment. According to the present invention, a “cocktail” of medicines or ingredients can successfully delay onset or progression of Alzheimer's Disease. In particular, a medical food cocktail composed of an inventive combination of standardized herbal extracts, vitamins, and minerals has been found to impact the biochemical and pathophysiological processes involved in Alzheimer's Disease.

The invention is a standardized cocktail, which includes, for example, extracts of tumeric, green tea, black pepper and vitamins and other nutritive ingredients. The cocktail affects behavioral and biochemical markers and immunohistochemistry related to neurodegeneration, as demonstrated in, for example, a novel transgenic mouse model of Alzheimer's Disease.

In one aspect, the invention provides a composition comprising curcumin, piperine, epigallocatechin-3-gallate and N-acetylcysteine. In some embodiments, the ingredients are provided in such amounts that the composition is therapeutically effective to treat a cognitive or neurological disorder in a patient. These amounts can be, for example, at least about 75 mg curcumin, at least about 0.6 mg piperine, at least about 35 mg epigallocatechin-3-gallate, and at least about 32 mg N-acetylcysteine. The cognitive or neurological disorder can be, for example, Alzheimer's disease, amyotrophic lateral sclerosis (ALS), mild cognitive impairment, frontotemporal dementia with Parkinsonism linked to chromosome 17, Pick's disease, progressive supranuclear palsy, or corticobasal degeneration. In some embodiments, the composition can also include one or more of α-lipoic acid in an amount of at least about 19 mg; vitamin B₁ in an amount of at least about 3 mg; vitamin B₆ in an amount of at least about 6 mg; vitamin B₁₂ in an amount of at least about 0.02 mg; folate in an amount of at least about 0.04 mg; and/or vitamin C in an amount of at least about 24 mg. In some embodiments, the composition includes all of these ingredients.

In another aspect, the invention provides methods of treating a cognitive or neurological disorder in a patient. The methods can comprise administering to the patient a therapeutically effective amount of a composition comprising curcumin, piperine, epigallocatechin-3-gallate and N-acetylcysteine. The composition can comprise, for example, at least about 1.05 mg/kg patient body weight curcumin, at least about 0.01 mg/kg patient body weight piperine, at least about 0.5 mg/kg patient body weight epigallocatechin-3-gallate, and at least about 0.4 mg/kg patient body weight N-acetylcysteine. In some embodiments, the composition can comprise, for example, one or more of α-lipoic acid in an amount of at least about 0.2 mg/kg patient body weight; vitamin B₁ in an amount of at least about 0.05 mg/kg patient body weight; vitamin B₆ in an amount of at least about 0.09 mg/kg patient body weight; vitamin B₁₂ in an amount of at least about 0.0002 mg/kg patient body weight; folate in an amount of at least about 0.0006 mg/kg patient body weight; and/or vitamin C in an amount of at least about 0.35 mg/kg patient body weight. In some embodiments, the composition can comprise all of these ingredients. The cognitive or neurological disorder treated can be, for example, Alzheimer's disease, ALS, mild cognitive impairment, frontotemporal dementia with Parkinsonism linked to chromosome 17, Pick's disease, progressive supranuclear palsy, and/or corticobasal degeneration. The composition can be administered in unit dosage form comprising one or more unit dosages daily.

In some embodiments, treating the cognitive or neurological disorder comprises treating one or more adverse cognitive symptoms associated with the cognitive or neurological disorder, such as, for example, memory loss, personality change, agitation, disorientation, loss of coordination, and/or inability to care for one's self. Treating the cognitive or neurological disorder can also comprise, for example, treating one or more adverse physiological symptoms associated with the cognitive or neurological disorder, such as amyloid plaques, tau protein tangles, tau protein phosphorylation, microtubule destabilization, and/or synaptic loss. In some embodiments, treating the cognitive or neurological disorder comprises reducing a level of a low molecular weight oligomeric beta amyloid peptide, such as Aβ*56, in the patient, in some cases by as much as or at least about 50%.

### BRIEF DESCRIPTION OF THE DRAWINGS

FIG. 1 is a schematic drawing of the pathophysiological processes involved in Alzheimer's disease.

FIG. 2 presents a series of graphs depicting the effects of a cocktail according to the present invention on performance in the Morris water maze tests in the Tg2576 transgenic mouse model.

FIG. 3 presents a series of graphs depicting the effects of a cocktail according to the present invention on novel object recognition in the Tg2576 transgenic mouse model.

FIG. 4 presents a series of graphs depicting the effects of a cocktail according to the present invention on several biochemical markers related to the etiology of AD in the Tg2576 transgenic mouse model.

FIG. 5 presents a series of graphs depicting the effects of a cocktail according to the present invention on performance in the Morris water maze (A-B) and on levels of several biochemical markers related to the etiology of AD (C-D) in the 3×Tg-AD mouse model.

### DETAILED DESCRIPTION

Embodiments of the invention are discussed in detail below. In describing embodiments, specific terminology is employed for the sake of clarity. However, the invention is not intended to be limited to the specific terminology so selected. A person skilled in the relevant art will recognize that other equivalent parts can be employed and other methods developed without parting from the spirit and scope of the invention. All references cited herein are incorporated by reference as if each had been individually incorporated.

In some embodiments, the invention provides a medical food cocktail that can slow, halt or reverse the development of Alzheimer's disease or another neurological or cognitive disorder during the early stages of the disease. The cocktail is composed of nutritional ingredients that are demonstrated to beneficially impact cognitive function and/or the biochemical or physiological processes thought to be involved in Alzheimer's disease. These ingredients are all currently listed as Generally Recognized As Safe (GRAS) by the FDA, or are self-affirmed as GRAS ingredients, or in common use as dietary supplements. The inventive compositions have been found to be beneficial in preventing, reducing the severity of, or reversing various neurological diseases or cognitive disorders, including but not limited to Alzheimer's disease, Parkinson's disease and mild cognitive impairment.

“Treat” refers to preventing, curing, reversing, attenuating, alleviating, minimizing, suppressing or halting at least one of the symptoms or deleterious effects of the diseases, disorders or conditions described herein. Treatment encompasses both therapeutic treatment and prophylactic or preventative measures. Those in need of treatment include those already with the disorder as well as those in which the disorder is to be prevented. Hence, the patient to be treated may have been diagnosed as having the disorder or may be predisposed or susceptible to the disorder.

“Effective” or “therapeutically effective” means sufficient to cause at least one of a patient's symptoms to decrease in frequency and/or intensity. The symptoms that are decreased in frequency and/or intensity can include, for example, one or more adverse cognitive or physiological symptoms.

“Cognitive or neurological disorder” encompasses any disorder that would be effectively treated by the compositions or methods of the present invention. For example, “cognitive or neurological disorder” includes Alzheimer's disease, ALS, mild cognitive impairment, frontotemporal dementia with Parkinsonism linked to chromosome 17, Pick's disease, progressive supranuclear palsy, or corticobasal degeneration, or any symptom or symptoms associated with these or other disorders.

“Administer” means to deliver one or more doses of one of the compositions to a patient. The methods of the present inventions can involve administration of the composition by any means and via any route of administration that is consistent with effective treatment of one or more of the diseases described herein. For example, the methods can involve administering the compositions orally.

The “patient” according to the present invention is a mammal, such as a human, which is diagnosed with one of the diseases, disorders or conditions described herein, or is predisposed to at least one type of the diseases, disorders or conditions described herein. The compositions of the present invention can be administered to any mammal that can experience the beneficial effects of the compositions and methods of the invention. Any such mammal is considered a “patient.” Such patients include humans and non-humans, such as humans, domestic and farm animals, and zoo, sports, or pet animals, such as dogs, horses, cats, cows, mice, rats, etc.

“Adverse cognitive symptom” encompasses any cognitive symptom that can be effectively treated by the compositions and methods of the present invention. Examples of adverse cognitive symptoms include, without limitation, memory loss, personality change, agitation, disorientation, loss of coordination, and inability to care for one's self.

“Adverse physiological symptom” encompasses any physiological symptom that can be effectively treated by the compositions and methods of the present invention. Examples of adverse physiological symptoms include, without limitation, formation or accumulation of amyloid plaques, formation or accumulation of tau protein tangles, tau protein phosphorylation, microtubule destabilization, and/or synaptic loss.

S-adenosylmethionine (SAMe), a biomolecule made from an amino acid molecule and ATP, is a substance that occurs naturally in the body. It plays a role in 35-40 biochemical reactions. In most people, the body can make all the SAMe it needs, but some patients with depression and other psychological conditions have been found to have lower levels of the compound, as well as lower levels of folate and vitamin B₁₂. Each of these substances plays a part in the metabolic process of “methyl donation,” or “methylation,” a process in which a methyl group is attached to a protein or lipid molecule. These methylation reactions are involved in the production of the neurotransmitters serotonin and dopamine in the brain as well as the activation of enzymes that help repair joints and the liver. There is evidence that serotonin is a factor in migraine and is involved in the so called “rebound effect,” because of its vasoconstricting effect at elevated levels and subsequent vasodilation as levels decrease. Folate deficiency also appears to reduce brain serotonin and contribute to depression in individuals. Folate supplementation can contribute to the achievement of an appropriate balance between serotonin generation and breakdown, which can lead to a decrease in the incidence of depression as well as a minimization of the cycling between vasodilation and vasoconstriction caused by fluctuations in serotonin levels.

Several markers and/or chemical processes have been identified that either contribute to the development of neurological or cognitive deficiencies, particularly AD, or are present in higher amounts in individuals diagnosed with AD. These are referred to herein as AD Factors. However, these factors are not limited to Alzheimer's and are found in various other neurological and cognitive deficiencies as well. Several active compounds can be used to address these AD Factors. The use of a cocktail or mixture of these active compounds to prevent, slow or reverse the progression of Alzheimer's Disease, Parkinsons, ALS, mild cognitive impairment, and other types of dementia or neurological deterioration can address the multiple factors associated with the etiology or progression of these diseases. As used herein, “cocktail” and combination” or “combination diet” can be used interchangeably. Compositions according to the present invention can prevent, slow the progression of or reverse the effects of AD and other disorders, such as dementia, in human subjects. The inventive compositions can also reduce the levels of the biochemical markers and/or the frequency of biochemical events that are associated with the etiology of AD and other disorders.

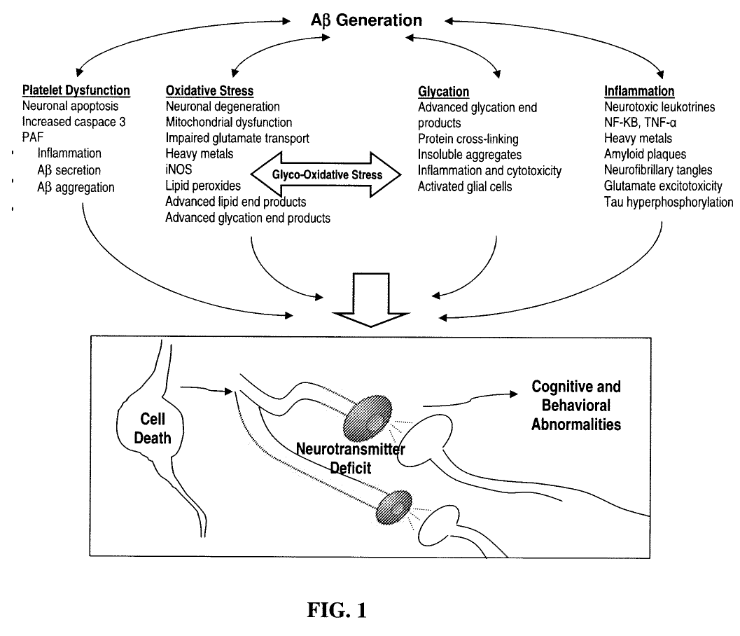

The compositions and methods of the present invention have a beneficial impact on one or more of at least four major biochemical phenomena or pathways: inflammation, oxidative stress, glycation/dysinsulinemia, and platelet function (see, e.g., FIG. 1). The compositions also beneficially affect a key marker, homocysteine levels, that is important contributor to the development or progression of AD.

An additional factor, acetylcholinesterase inhibition, is addressed by currently existing drugs (Aricept™ (donepezil), Exelon™ (rivastigmine) and Reminyl™ (galantamine). Additionally, Namenda™ (memantine) is used to prevent toxic levels of glutamate, also a chemical messenger, in the brain. The four pathways and their associated mechanisms, markers and factors are also set forth in Table 1. Several naturally occurring compounds or groups of compounds have been shown to decrease, reverse or prevent these phenomena from occurring. Applicant has surprisingly discovered that there is a synergistic benefit in combining three or more of these compounds into a cocktail. The compositions and methods of the present invention address several of the different mechanisms that contribute to the onset or progression of AD, as well as other neurological deficiencies and diseases. Furthermore, the combination creates an environment where it is difficult for beta-amyloid plaques to either develop or deposit.

TABLE 1
AD Associated Mechanisms, Markers and Factors

| Oxidative Stress | Glycation | Inflammation | Platelet Function |
|---|---|---|---|
| Mitochondrial | Matrix | Tau and β amyloid | Secretion of Abeta |
| dysfunction | metalloproteinases | MMP excitation | Glutamate |
| Glutamate transport | (MMP) production | Homocysteine | Inflammation |
| Beta amyloid | Oxidation | Heavy metals | PAF |
| Induced nitric oxide | Inflammation | Mitochonrial | Aggregation |
| synthase | β-Amyloid toxicity | dysfunction | +Capsase 3 |
| Heavy Metals | Cognition | TNF-β, NF-KB | |
| Ubiquitin- | Mitochondrial | Platelet activation | |
| Proteosome. | dysfunction | factor | |
| Heat shock protein | Advanced glycation | | |
| Platelet activation | end products | | |
| factor | Pentosidine, N^{ε}- | | |
| Cognition | carboxymethyllysine | | |
| TBARS | (CML) | | |
| Malondialdehye | | | |
| 4-Hydroxnonenal | | | |

While a single cause for Alzheimer's disease has not been identified, the brain of people diagnosed with AD typically exhibit sticky plaques composed of β amyloid protein deposits (plaques or amyloid plaques) as well as tau protein tangles resulting from hyperphosphorylation of tau proteins. The compositions and methods of the present invention bring about the prevention of plaques and tangles, and/or their reversal/reduction if formed. Compositions according to the invention have been shown to reduce amyloid plaques, tau protein tangles, microtubule destabilization, synaptic loss, and tau protein phosphorylation, and to treat memory loss, personality change, agitation, disorientation, inability to care for one's self, and loss of coordination in AD patients.

Beta amyloid (Aβ or Abeta) peptides have been the central focal point of AD research for over a decade, and is generally considered as the upstream causative factor. The strongest evidence for this position derives from molecular genetic studies of the three genes—amyloid precursor protein (APP), presenilin 1 (PS1) and presenilin 2 (PS2) that underlie some AD cases, as they all modulate some aspect of Aβ metabolism, increasing the propensity for AB to aggregate. In addition, the apolipoprotein E gene, which is a modifier gene linked to late-onset disease, affects the rate of AB aggregation. Neurofibrillary tangles are filamentous inclusions that accumulate in selective neurons in the brains of individuals with AD, but they also occur in other neurodegenerative disorders including frontotemporal dementia with Parkinsonism linked to chromosome 17 (FTDP-17), Pick's disease, progressive supranuclear palsy (PSP), and corticobasal degeneration (CBD). The major component of tangles is the microtubule-associated protein tau. In its normal state, tau is a soluble protein whose function is to promote microtubule assembly and stabilization. Pathological tau protein, by contrast, exhibits altered solubility properties, forms filamentous structures, and is abnormally phosphorylated at certain residues. Phosphorylated tau shows reduced affinity for microtubules.

The tau protein is encoded by a single gene (MAPT) located on chromosome 17, although it is alternatively spliced to yield 6 major protein isoforms in the adult human brain. The tau gene contains 15 exons, and exons 2, 3, and 10 can be alternatively spliced. Four imperfect tandem repeats are encoded by exons 9-12, hence, alternative splicing of exon 10 yields isoforms with 3 or 4 repeat domains (3R and 4R tau), depending if exon 10 is absent or present, respectively. Alternative splicing of exons 2 and 3 yields variants containing zero (0N), one (1N), or two (2N) inserts at the amino terminus, such that 6 tau isoforms are formed: 3R0N, 3R1N, 3R2N, 4R0N, 4R1N, and 4R2N. In the adult human brain, the proportion of 3R to 4R tau is 1:1, whereas in the adult mouse brain, 4R tau is the only tau isoform present. Tauopathies can be further classified based on whether tangles are comprised of 3R or 4R tau isoforms. For example, in AD, both 3R and 4R tau accumulate in neurofibrillary tangles; other disorders are marked by only 3R tau (e.g., Pick's disease) or 4R tau (e.g., CBD and PSP). In AD, tau pathology is restricted to neurons, but in certain other tauopathies, such as 4R tauopathies CBD and PSP, tau inclusions are also observed in glia.

Effective treatments for AD will address, for example, the aggregating pathologies and/or the loss of synapses and neurons. One approach to treatment is to prevent the development of pathologies in the first place, or at least delay the onset of the disease.

The following discusses four major biochemical phenomena or pathways that are implicated in the etiology of AD: inflammation, oxidative stress, glycation/dysinsulinemia, and platelet function.

Inflammation—Chronic inflammation has been observed to damage host tissue, and brain neurons are particularly vulnerable. Inflammatory mediators can be produced and elevated in affected regions of brains of individuals with AD. Non-immune mediated chronic inflammatory responses in brain parenchyma, which can occur in response to the production of beta amyloid (Aβ or Abeta) peptides, are believed to be involved in AD progression. Neurodegenerative plaque formation in AD is characterized by the up-regulation of interleukin (IL)-1 and interleukin-6, and this up-regulation can play a role in the pathogenesis of AD. Advanced glycation end products have been shown to exert an inflammatory effect as well. In some embodiments, the compositions and methods of the invention use naturally occurring compounds to slow or halt the chronic inflammatory-like process that occurs in the early pathological cascade of AD. Markers of inflammatory response include serum alpha (1) anti-chymotrypsin, nuclear factor (NF)-kappaBeta, high sensitivity C-reactive protein, platelet activation factor, transforming growth factor beta, tumor necrosis factor (TNF)-alpha and inflammatory cytokine production in general. An inflammatory cascade precipitated by the formation of Abeta plaques in the brain is thought to be a prime cause of neuronal death. The inflammatory marker C-reactive protein and microglial inflammatory markers, such as the inflammatory cytokines IL-1β and IL-6 and the inflammatory proteins nitric oxide synthase-2 (NOS2) and TNF-alpha, are all up-regulated in tissue from Alzheimer's patients. C-reactive protein-like inflammation has been demonstrated in both the senile protein plaques (polymorphous beta-amyloid protein deposits found in the brain in Alzheimer disease and normal aging) and neurofibrillary tangles of Alzheimer's victims. Chronic inflammation may also be responsible for the degeneration of the hippocampus, a particularly vulnerable part of the brain. Compositions of the the invention, such as those comprising naturally occurring compounds, such as phytochemicals, that have a beneficial impact on inflammation, can contribute to the prevention of AD and the slowing of its progression, especially because many of these processes are measurable long before clinical symptoms appear.

Oxidative Stress—Oxidative stress is caused by an imbalance between the production of reactive oxygen species (many of which are free radicals) and a biological system's ability to readily detoxify the reactive intermediates or easily repair the resulting damage. Alzheimer's patients also exhibit high serum levels of markers of oxidative stress and low plasma levels of antioxidants and free radical scavengers. Like inflammation, oxidative stress can play a role in the development and progression of most chronic degenerative diseases, including AD. Alzheimer's-diseased brains are characterized by excessive Abeta deposition and by extensive oxidative stress. There are several sources of oxidative stress, including advanced glycation end products, microglial activation and the sequelae of Abeta. Membrane permeable antioxidants prevent the up-regulation of induced nitric oxide synthase (iNOS), and some can be viewed both as antioxidants and as anti-inflammatory drugs. The destructive free radicals produced by oxidative stress can damage sensitive neurons. Metals such as iron, copper, zinc, and aluminum exacerbate the production of free radicals, as does the presence of Abeta plaques, creating a vicious cycle of neuronal damage. Nutritional antioxidants can block or reduce neuronal death. Compositions of the present invention, which can include, for example, antioxidants, can contribute to preventing and/or slowing AD. Of particular interest are combinations of antioxidants that have complementary or synergistic activity, or that quench several types of reactive oxygen species.

Glycation/Dysinsulinemia—Glycation is the non-enzyme-mediated, generally haphazard reaction of protein or lipid molecules with sugars in a way that interferes with the activity of the protein or lipid. Glycation is the first step in the production of advanced glycation end-products (AGEs), which are a major cause of the physical manifestations of aging and damage to tissue elasticity. Extracellular AGEs can accumulate in the Abeta plaques of Alzheimer's patients, causing further oxidative stress on the surrounding neural tissue. AGEs are also found in the serum and cerebral spinal fluid of Alzheimer's patients. An increasing percentage of adults and children are overweight, and obesity often causes dysinsulinemia, which can lead to increased glycation of proteins. The incidence of non-insulin dependent diabetes mellitus (NIDDM) is increasing, even in people within normal body mass indicies (BMIs). This trend, coupled with the potential effects of glycation on all types dementia, is of concern. Glycoxidative (glycation+oxidation) stress creates a cascade of events leading to neurodegeneration, such as that found in AD. The accumulation of AGEs explains neuro-pathological and biochemical events such as protein cross linking, free radical damage, neuronal apoptosis and glial activation, all of which are features of AD. Several markers of glycoxidative stress have been identified. Examples of these markers are pentosidine, N(epsilon)-(carboxymethyl)lysine (CML), fructosamine, malondialdehyde (MDA), and 4-hydroxy-2-noneal (HNE). The compositions of the present invention, which can include, for example, AGE inhibitors, can slow, halt or reverse glycoxidative effects on AD.

Platelet Function—Platelets are a source of beta-amyloid precursor protein. Increased platelet activation, abnormal platelet function and increased circulating beta-amyloid have been observed in AD. Activated platelets are a source of Abeta peptides, and beta-amyloid tends to aggregate platelets and support their adhesion. Non-steroidal anti-inflammatory drugs (NSAIDs) can reduce the inflammatory response of microglial cells. A significant correlation exists between platelet activating factor (PAF) binding and degree of cognitive impairment in Alzheimer's patients. Similarly, neurons pretreated with PAF antagonists were resistant to damage by Abeta and also exhibited a reduced activation of caspase-3, a marker of apoptosis (i.e., programmed cell death). Compositions of the present invention, hich can include, for example, ingredients that have anti-inflammatory effects and/or that normalize platelet function, can be beneficial as therapeutic options in AD.

Homocysteine—Homocysteine presence or absence is believed to be a marker of, and/or a risk factor for, both stroke and cardiovascular disease. It has been estimated that exceeding normal levels (5-15 micromol/L) by as little as 5 micromol/L increases the risk of coronary artery disease by 60 percent in men and 80 percent in women. High homocysteine levels are also a risk factor for Alzheimer's disease. Individuals with a blood plasma homocysteine level above 14 micromol/L have been found to have nearly twice the risk of developing Alzheimer's disease as do people with lower levels. A 5 micromol/L increase in homocysteine level has been found to correspond to a 40 per cent increased risk of developing Alzheimer's disease. Also, this damage can be halted and even reversed by repair of nerve cell DNA damage in the brain.

The Alzheimer's Cocktail Components

In some embodiments, the invention provides compositions for use in preventing, treating or reducing the severity of AD. The compositions can comprise, for example, a combination of two, three or more of curcumin, alpha lipoic acid, N-acetylcysteine, Vitamins C and E, epigallocatechin-3-gallate (from green tea extract), one or more B-complex vitamins (B₁, B₅, B₆, B₁₂ and folate), L-camosine, protolytic enzymes and piperine. Table 2 lists the AD Factors and the compounds proposed to address each. Several of these compounds address more than one of these factors. In addition, several of the ingredients have been shown to exhibit antocholinesterase activity. There are no known maximum daily dosage levels for many of these compounds, and many are not toxic unless consumed in very high quantities. All are generally recognized to be safe for daily consumption.

TABLE 2
Medical Food Cocktail Ingredients
and Disease Processes Targeted

| | Effect on Biochemical Processes? | | | |
|---|---|---|---|---|
| Cocktail | Oxidative | | | Platelet |
| Ingredient | Stress | Inflammation | Glycation | Function |
| Curcumin | * | * | * | * |
| Piperine | * | * | * | * |
| Epigallocatechin- | * | * | | * |
| 3-gallate | | | | |
| (EGCG) | | | | |
| α-Lipoic Acid | * | * | | * |
| N-Acetylcysteine | * | * | * | |
| B Vitamins | | | | |
| B₁ | | | * | |
| B₆ | * | * | * | * |
| B₁₂ | * | * | | |
| Folate | * | | | |
| Vitamin C | * | * | | |
| Vitamin E | * | * | | * |

In some embodiments, the compositions of the present invention are to be administered at a dosage of from about 3 mg/kg/day to about 200 mg/kg/day. In some embodiments, the compositions of the present invention can be administered in a dosage of about 200 mg/day to about 15,000 mg/day. The dosage to be administered can comprise, for example, curcumin in an amount of from about 1.05 to about 85 mg/kg patient body weight, or from about 8.8 to about 13.4 mg/kg body weight, or from about 11.1 to about 111 mg/kg patient body weight, or from about 88.8 to about 133.2 mg/kg patient body weight; piperine in an amount of from about 0.01 to about 1.0 mg/kg patient body weight, or from about 0.09 to about 0.9 mg/kg patient body weight or from about 0.09 to about 0.11 mg/kg patient body weight, or from about 0.7 to about 1.1 mg/kg patient body weight; EGCG in an amount from about 0.5 to about 40 mg/kg patient body weight, or from about 44 to about 66 mg/kg patient body weight, or from about 5.5 to about 55 mg/kg patient body weight, or from about 4.4 to about 6.6 mg/kg patient body weight; and N-acetylcysteine in an amount from about 0.4 to about 35 mg/kg patient body weight, or from about 37 to about 56.4 mg/kg patient body weight, or from about 4.7 to about 47 mg/kg patient body weight, or from about 3.7 to about 5.7 mg/kg patient body weight. As used herein, “about” may refer to a range from 10% below the referenced number to 10% above the referenced number. For example, “about 50” may mean from 45 to 55. Other meanings of “about” may be apparent from the context.

Curcumin is a polyphenol that comprises the active component of the plant/spice referred to as turmeric (Curcuma longa). The root and rhizome of turmeric have been used medicinally. The plant extract is standardized to 90-95% curcumin or curcuminoids.

Curcumin is a strong antioxidant, is a potent inhibitor of lipid peroxidation and has several anti-inflammatory effects. For example, curcumin is thought to bring about decreased histamine levels, increased natural cortisone production by the adrenals, and modified synthesis of specific interleukins, cytokines, leukotrienes and eicosanoids in general. Curcumin can modulate many inflammatory markers, such as TNF-a and NF-Kappa-b. It also provides hepatoprotective benefits against a number of toxic compounds. Curcumin also demonstrates anti-platelet effects, which may protect against beta amyloid-induced platelet aggregation and platelet adhesions. It also has anti-glycation effects and can decrease levels of platelet-activating factor (PAF), thus disrupting normal platelet function. Curcumin protects normal human umbilical vein endothelial cells from Abeta. In studies on mice, low doses of curcumin significantly lowered levels of oxidized proteins and IL-1 beta in mice brains. It also has been shown to suppress Abeta-induced cognitive defects and oxidative damage. According to the present invention, compositions comprising low dose curcumin can decrease insoluble Abeta, soluble Abeta, and plaque burden by, for example, 43-50%.

In Alzheimer transgenic mice, dietary curcumin is associated with decreased levels of oxidized proteins and interleukin-1 beta. A suppression of microgliosis has also been observed. In addition, curcumin prevents the accumulation of advanced glycation endproducts in diabetic rats receiving dietary curcumin (200 mg/kg body weight) compared to control diabetic rats without curcumin. It also brings about a significant reduction in lipid peroxidation products (which are indicators of oxidative stress) in the curcumin fed rats.

Compositions according to the present invention comprising, for example, curcumin may be effective in the prevention and treatment of Alzheimer's disease. Curcumin inhibits both the formation and growth of beta-amyloid fibrils from Abeta in a dose-dependent manner. Curcumin also inhibits neuroglial proliferation in rats. In a neuroblastoma cell line, curcumin inhibited activation of the inflammatory marker nuclear factor kappa-beta (NFKB). Likewise, curcumin inhibits inflammation-related cyclooxygenase-2 gene expression in microglial cells. Curcumin also inhibits platelet activating factor (PAF) and platelet aggregation induced by platelet agonists. Curcumin acts as a metal chelator, thus reducing Abeta aggregation and toxicity, while suppressing damage from inflammation. Along this line, curcumin has been shown to chelate both cadmium and lead in rat brain homogenates, protecting against lipid peroxidation. Supplementation with tumeric reduces oxidative stress and attenuates the development of fatty streaks in rabbits fed a high cholesterol diet. Curcumin can be administered in daily dosages of, for example, from about 250 mg to about 10 grams, or from about 250 mg to about 5 grams, or from about 500 mg to about 5 g, or about 1000 mg.

Alpha lipoic acid (ALA), a disulfide, is an antioxidant that is both lipid- and water-soluble. It promotes synthesis of the endogenous antioxidant glutathione. ALA can enhance glucose uptake, inhibit glycosylation and alleviate peripheral neuropathies and associated nerve pain. ALA prevents AGE induced increases in NF-kappa-b activation, thus protecting against endothelial dysfunction. ALA stabilizes cognitive function in elderly, beginning-stage Alzheimer patients. We have investigated the potential effectiveness of alpha-lipoic acid (ALA) against cytotoxicity induced by Abeta peptide (30 microM) and hydrogen peroxide (H₂O₂) (100 microM) with the cellular 3-(4,5-dimethylthiazol-2-yl)-2,5-diphenyl-tetrazolium bromide (MTT) reduction and fluorescence dye propidium iodide assays in primary neurons of rat cerebral cortex. It was found that treatment with ALA protected cortical neurons against cytotoxicity induced by Abeta or H₂O₂. (Zhang L, Xing G Q, Barker J L, Chang Y, Maric D, Ma W, Li B S, Rubinow D R. Alpha-lipoic acid protects rat cortical neurons against cell death induced by amyloid and hydrogen peroxide through the Akt signalling pathway. Neurosci Lett 2001; 312:125-8.)

AGEs have been shown to induce lipid peroxidation in a neuronal cell line in a dose-dependent manner. Blocking the specific AGE-receptor RAGE can reduce the AGE-mediated formation of lipid peroxidation products. Similar effects have been shown by administration several antioxidants, such as alpha-lipoic acid, N-acetylcysteine, 17 beta-estradiol and/or aminoguanidine. Extracellularly administered alpha-lipoic acid reduces AGE-albumin-induced endothelial expression of vascular cell adhesion molecule-1 (VCAM-1) and monocyte binding to endothelium in vitro, and has also demonstrated significant antioxidant potential. ALA can be administered in daily dosages of, for example, from about 50 mg to about 2 grams, or from about 100 mg to about 1 gram, or from about 150 mg to about 500 mg, or about 300 mg. For example, the R form of ALA can be administered.

N-Acetylcysteine (NAC) administration has been studied in patients who met National Institute of Neurological and Communicative Disorders and Stroke-Alzheimer's Disease and Related Disorders Association criteria for probable AD. NAC was administered in a double-blind fashion, and testing for efficacy occurred after 3 and 6 months of treatment. NAC treatment achieved beneficial results on nearly every outcome measure, although significant differences were obtained only for a subset of cognitive tasks.

Oxidative stress may play a crucial role in age-related neurodegenerative disorders. The ability of the two antioxidants, ALA and NAC, to reverse the cognitive deficits found in the SAMP8 mouse has been examined. By 12 months of age, this strain of mouse develops elevated levels of Abeta and severe deficits in learning and memory. Twelve-month-old SAMP8 mice, in comparison with 4-month-old mice, had increased levels of protein carbonyls (an index of protein oxidation), increased readings in the thiobarbituric acid reactive species (TBARS) assay (an indicator of lipid peroxidation) and a decrease in the weakly immobilized/strongly immobilized (W/S) ratio of the protein-specific spin label MAL-6 (an index of oxidation-induced conformational changes in synaptosomal membrane proteins). Chronic administration of either ALA or NAC improved cognition of 12-month-old SAMP8 mice in both the T-maze footshock avoidance paradigm and the lever press appetitive task without inducing non-specific effects on motor activity, motivation to avoid shock, or body weight. These effects are believed to have occurred directly within the brain, as NAC crossed the blood-brain barrier and accumulated in the brain. Furthermore, treatment of 12-month-old SAMP8 mice with ALA reversed all three indexes of oxidative stress. These results support the hypothesis that oxidative stress can lead to cognitive dysfunction, and they also provide evidence for a therapeutic role for antioxidants. NAC has also been shown to antagonize N-methyl-D-aspartate (NMDA) caused glutamergic excitation and its neurotoxicity. NAC can be administered in daily dosages of, for example, from about 100 mg to about 2 grams, or from about 250 mg to about 1500 mg, or from about 250 mg to about 1000 mg, or about 500 mg.

Vitamins C and E are well known for their anti-oxidant properties. In a study of more than 4740 subjects in Cache County, Utah, use of vitamin C and E supplements was associated with a significant reduction in risk of Alzheimer's disease. Similar results were seen in the Honolulu-Asia aging study of 3385 elderly men, in which vitamin C and E supplementation was associated with a protective effect for vascular and mixed dementia. Dementia patients and Alzheimer's patients also exhibit lower plasma vitamin C concentrations than control subjects with no cognitive impairment. Vitamin E prevents increased protein oxidation, reactive oxygen species, and Abeta-induced neurotoxicity in a rat embryonic hippocampal neuronal culture. Likewise, in a rat model of traumatic brain injury (a risk factor for Alzheimer's), rats treated with vitamin E exhibited no increase in Abeta peptides or cognitive dysfunction, in contrast to rats not receiving vitamin E. A daily dosage can include, for example, at least about 100 mg of vitamin C as ascorbic acid or dehydroascorbic acid, or from about 100 mg to about 2 g, or from about 100 mg to about 1 g, or about 300 mg. A daily dosage can include, for example, from about 100 to 1000 IU of vitamin E, or from about 100 to about 800 IU, or from about 200 to about 600 IU, or about 400 IU. For example, vitamin E can be administered in the form of D-alpha tocopherols, tocopheryls, or a combination of these and other vitamin E isomers.

L-Carnosine (b-alanyl-L-histidine) is a naturally occurring di-peptide of the amino acids alanine and histidine. It is found in brain, muscle and other innervated tissues. High concentrations of carnosine are present in long-lived cells such as neuronal tissues and may be an aging marker. Camosine, a powerful antioxidant, is active against by-products and metabolites of reactions with reactive oxygen species, and it also has an anti-glycosylation effect. MDA (malondialdehyde), a marker of DNA damage from oxidative stress, is blocked by carnosine.

Carnosine prevents sugar aldehydes from reacting with the amino acids in protein molecules, and also reverses the process. Carnosine's protection against cross-linking and the formation of abnormal AGEs, and its ability to reduce or prevent cell damage caused by beta amyloid, provide anti-aging benefits. In an 8-week study using L-camosine, children with autistic spectrum disorders showed statistically significant improvements on the Gilliam Autism Rating Scale (total score and the Behavior, Socialization, and Communication subscales) and the Receptive One-Word Picture Vocabulary test (all p<0.05). Improved trends were noted on other outcome measures. Although the mechanism of action of L-camosine is not well understood, it may enhance neurologic function, perhaps in the enterorhinal or temporal cortex. L-camosine can be administered in daily dosages of, for example, at least about 100 mg.

Epigallocatechin-3-gallate (EGCG), a polyphenol commonly recovered from green tea extract, which is standardized to a minimum of 50% EGCG, is a potent anti-inflammatory and antioxidant compound. EGCG is believed to be involved in amyloid precursor protein (APP) secretion and protection against toxicity induced by beta-amyloid. EGCG can decrease or prevent Abeta toxicity in P C12 cells. Green tea can improve age-related cognitive decline and confer neuroprotection in Alzheimer's disease models. Although initially ascribed to the antioxidant properties of green tea, the neuroprotective effects may be due to a wide spectrum of cellular signaling events targeting many disease processes.

In cultured hippocampal neurons exposed to Abeta for a 48-hour period, co-treatment of the cells with EGCG decreased the levels of malondiadehyde (a marker for glycation) and caspase C (a marker of abnormal platelet function) compared to controls with no EGCG. Cells treated with EGCG also exhibited increased survival compared to contols. Similarly, a water-based extract of green tea inhibited the aggregation of rabbit platelets in vitro. The investigators found that green tea was comparable to aspirin in preventing platelet aggregation. Finally, EGCG was shown to inhibit the inflammatory markers TNF-a and NF-KB, as well as interleukin-1 proinflammatory signal transduction in cultured epithelial cells. It also appears that EGCG may protect against ischemic neuronal damage. EGCG can be administered in daily dosages of, for example, from about 10 mg to about 3000 mg, or from about 10 mg to about 1000 mg, or from about 50 mg to about 500 mg, or about 100 mg.

Complex vitamins (such as B₆, B₁₂ and folate) can prevent or reduce homocysteine (HC) damage. Elevated HC levels induce direct neurotoxicity and potentiate Abeta and glutamate neurotoxicity. The B vitamins may both improve cognitive functioning and reduce the levels of biochemical markers for Alzheimer's disease processes. In cultured brain cells grown in media deficient in folic acid, the addition of methotrexate (a folic acid inhibitor) to the media rendered nerve cells more susceptible to death from Abeta. Likewise, in a mouse model of Alzheimer's a folic acid-deficient diet resulted in DNA damage and damage to the hippocampus. In patients from the Framingham Heart Study, low levels of plasma B₆ were correlated with high levels of the inflammatory marker C-reactive protein. In patients with mild cognitive impairment and increased homocysteine levels, treatment with a B₆-B₁₂-folate combination improved blood brain barrier function and appeared to stabilize cognitive status. In in vitro studies, vitamin B₁ inhibited formation of advanced glycation end products in bovine serum albumin, ribonuclease A, and human hemoglobin. Low B₁₂ and folate blood levels are associated with dementia. Vitamins B₆, folate and B₁₂ can reduce these elevated HC levels. Vitamin B₅ (pantothenic acid) is also necessary to form acetylcholine. Additionally, certain lesser known metabolites or alternative forms of some of the B vitamins, such as B₁, B₆ and B₁₂, play important roles in AD beyond their identified uses for reduction of homocysteine. For example, the hydroxycobalamine form of B₁₂ has been found to scavenge NO radicals, which have been associated with neurodegeneration and migraines. The benfotiamine form of vitamin B₁, which is a preferred form of vitamin B₁ for use in the present invention, is a fat-soluble form of vitamin B₁ and has demonstrated significant benefit against excessive glycation and advanced glycation endproducts (AGEs), which have been associated with Abeta formation and glia inflammation. Furthermore, folate compositions have been found to address inflammation, for example that caused by NO, as well as endothelial function. Nitrogen oxide (NO) synthase creates NO which causes inflammation in tissue.

The beneficial properties of folates can also be enhanced by the concurrent use of certain B vitamins, particularly pyridoxal-5-phosphate (P5P) and hydrocobalamin, and antioxidants such as vitamin E, s-adenosylmethionine (SAMe) and coenzyme Q10 (CoQ1O). Addition of NO synthase inhibitors, such as amino-guanidine, L-camitine, asymmetric arginine, and certain plant derived phytochemicals, can enhance the inflammation reducing properties of folates. For example, a daily dosage can comprise 100 mcg to 10 mg, or from about 1 mg to about 10 mg, or from about 2 mg to about 8 mg, or about 5 mg folate. A daily dosage can also comprise one or more of from about 100 mcg to 10 mg, or from about 100 mcg to about 5 mg, or from about 100 mcg to about 2.5 mg, or about 1 mg, of the hydroxycobalamin or methylcobalamin form of vitamin B₁₂; from about 1 to about 200 mg, or from about 10 to about 100 mg, or from about 25 to about 100 mg, or about 50 mg of B₆ (pyridoxal-5-phosphate or pyridoxamine); from about 10 to 500 mg, or from about 10 to 250 mg, or from about 10 to 100 mg, or about 25 mg of vitamin B₁₂; and from about 10 mg to about 1,000 mg, or from about 10 to 500 mg, or from about 10 to about 100 mg, or about 25 mg of riboflavin (vitamin B₂).

Folic acid or salts thereof, referred to as folates, along with vitamins B₆ and B₁₂, are required in metabolic pathways involving methionine, homocysteine, cystathionine, and cysteine. The term “folates,” as used herein, is meant to include, at a minimum, folacin (USP folic acid), naturally occurring folinic acid, 5-methyl tetrahydrofolate, and tetra hydrofolate as well as salts or metabolites of these compounds. It appears that folate, B₆ and B₁₂ are all necessary for normal metabolism. However, these three compounds each function in a different manner. Folate, even if available at normal levels, is consumed in the metabolic process and therefore must be constantly replenished by diet or supplements. However, B₆ and B₁₂ function as co-factors. While necessary for the respective metabolic process to proceed, they are each regenerated. Therefore, if they are present in normal amounts in serum, supplementation may not be necessary. B₁₂ in the form of 5′-deoxyadenosylcobalamin is an essential cofactor in the enzymatic conversion of methylmalonylCoA to succinylCoA. The remethylation of homocysteine (HC) to methionine, which is catalyzed by methionine synthase, requires folate in the form of methyltetrahydrofolate and B₁₂ in the form of methylcobalamin. HC is condensed with serine to form cystathionine (CT) in a reaction catalyzed by cystathionine beta-synthase, which requires B₆ (pyridoxal phosphate). CT is also hydrolyzed in another B₆-dependent reaction to cysteine and alpha-ketobutyrate. Homocysteine is a modified form of the amino acid methionine, and it is tightly regulated by enzymes which require folate. By impairing DNA repair mechanisms and inducing oxidative stress, homocysteine can cause the dysfunction or death of cells in the cardiovascular and nervous systems. Homocysteine appears to be present in many disease states. However, dietary folate stimulates homocysteine removal and may thereby protect cells against disease processes.

The principal biochemical function of folates is the mediation of one-carbon transfer reactions. 5-methyltetrahydrofolate donates a methyl group to homocysteine in the conversion of homocysteine to L-methionine. The enzyme that catalyzes this reaction is methionine synthase. Vitamin B₁₂ is a cofactor in the reaction. This reaction is of great importance in the regulation of serum homocysteine levels. The L-methionine produced in the reaction can participate in protein synthesis and is also a major source for the synthesis of S-adenosyl-L-methionine (SAMe). The methyl group donated by 5-methyltetrahydrofolate to homocysteine in the formation of L-methionine is used by SAMe in a number of transmethylation reactions involving nucleic acids, phospholipids and proteins, as well as in the synthesis of epinephrine, melatonin, creatine and other molecules. Tetrahydrofolate is the folate-containing product of the methionine synthase reaction. 5-Methyltetrahydrofolate is generated by conversion of 5,10-methylenetetrahydrofolate into 5-methyltetrahydrofolate via the enzyme methyleneterahydrofolate reductase (MTHFR). 5,10-Methylenetetrahydrofolate is regenerated from tetrahydrofolate via the enzyme serine hydroxymethyltransferase, a reaction which, in addition to producing 5,10-methylenetetrahydrofolate, yields glycine.

In addition to its role in the metabolism of homocysteine, 5,10-methylenetetrahydrofolate supplies the one-carbon group for the methylation of deoxyuridylic acid to form the DNA precursor thymidylic acid. This reaction is catalyzed by thymidylate synthase and the folate product of the reaction is dihydrofolate. Dihydrofolate is converted to tetrahydrofolate via the enzyme dihydrofolate reductase.

Folates are also involved in reactions leading to de novo purine nucleotide synthesis, interconversion of serine and glycine, generation and utilization of formate, the metabolism of L-histidine to L-glutamic acid, the metabolism of dimethylglycine to sarcosine and the metabolism of sarcosine to glycine.

One of the natural folates, folinic acid, is used as a pharmaceutical agent. Folinic acid, which is also known as leucovorin, citrovorum factor or 5-formyltetrahydrofolate, is used as rescue therapy following high-dose methotrexate in the treatment of osteosarcoma. It is also used to diminish the toxicity of methotrexate. It is used in the treatment of megaloblastic anemia resulting from folate deficiency, and also in the prevention or treatment of the toxic side effects of trimetrexate and pyrimethamine. The combination of folinic acid and 5-fluorouracil has until recently been standard therapy for metastatic colorectal cancer. Folinic acid increases the affinity of flurouracil for thymidylate synthase. Folinic acid is available as a calcium salt for parenteral or oral administration.

Folic acid is also called pteroylglutamic acid or PGA. Its full chemical name is N-[4-[[(2-amino-1,4-di-hydro-4-oxo-6-pteridinyl)methyl]amino]benzoyl]-L-glutamic acid. Older names for folic acid are vitamin Bg, folicin, vitamin Bc and vitamin M. Its molecular formula is C₁₉H₁₉N₇O₆ and its molecular weight is 441.40 daltons. Folic acid forms yellowish-orange crystals. The color is imparted by the pteridine ring of folic acid. Pteridine also imparts color to butterfly wings.

Folate has been prescribed as a nutritional supplement for many medical conditions associated with elevated homocysteine levels. Folate supplements appear to reverse the elevated homocysteine levels. However, the elevated homocysteine level may be a result of inadequate supply or excessive consumption of folate and not the cause of the disease. It is clinically beneficial in such instances to provide folate supplements because individuals with elevated homocysteine levels appear to be at increased risk for cardiovascular disease and stroke, and neurodegenerative disorders such as Alzheimer's and Parkinson's diseases as well as neural tube defects, spontaneous abortion, placental abruption, low birth weight, renal failure, rheumatoid arthritis, alcoholism, osteoporosis, neuropsychiatric disorders, non-insulin-dependent diabetes and complications of diabetes, fibromyalgia and chronic fatigue syndrome. Moderate elevations of HC might be associated with increased risk for vascular disease (Ueland et al. (1992) in Atherosclerotic Cardiovascular Disease, Hemostasis, and Endothelial Function (Francis, Jr., ed.), Marcel Dekker, Inc., New York, pp. 183-236). However, folic acid deficiencies have also been associated with periphereal vascular disease and coronary disease in individuals with normal homocysteine levels (Bunout, D. et al “Low Serum Folate but Normal Homocysteine Levels in Patients with Atheroslerotic Vascular Disease and Matched Healthy Controls”, Nutrition 2000, 16, p. 434-8), suggesting that folates may have a protective effect that extends beyond maintaining normal homocysteine levels. In addition, moderate hyperhomocysteinaemia has been shown to be frequently present in cases of stroke and to be independent of other stroke risk factors (Brattstrom et al. (1992) Eur. J. Clin. Invest. 22:214-221).

It is not clear if the various disease states are caused by elevated homocysteine levels or the elevated homocysteine levels are caused by other factors which are the primary cause of the disease state and result in elevated levels of homocysteine. For example, it is also known that folate supplements are useful where B₁₂ deficiencies exist, but where homocysteine levels may not be elevated. Individuals with B₁₂ deficiency can display neurologic disorders, typically relating to underlying anemia. However, supplementing diet with only folate is not medically recommend as these folate supplements may mask the underlying B₁₂ problem. U.S. Pat. No. 4,945,083, issued Jul. 31, 1990 to Jansen, entitled Safe Oral Folic Acid-Containing Vitamin Preparation, describes an oral vitamin preparation comprising the combination of 0.1-1.0 mg B₁₂ and 0.1-1.0 mg folate for the treatment or prevention of megaloblastic anemia.

Normal serum folate levels in healthy individuals are 2.5-20 ng/ml, with levels less than 2.5 ng/ml indicating the possibility of clinically significant deficiency. Like B₁₂ serum levels, however, serum folate levels are a relatively insensitive measure in that only 50-75% of patients with folate deficiency have levels less than 2.5 ng/ml, with most of the remaining 25-50% being in the 2.5-5.0 ng/ml range (Allen (1991), Cecil Textbook of Medicine, 19th Ed.).

A series of patents to Allen et al, (U.S. Pat. No. 5,563,126, U.S. Pat. No. 5,795,873, U.S. Pat. No. 6,207,651, U.S. Pat. No. 6,297,224 and U.S. Pat. No. 6,528,496)) teaches the use of oral compositions or a transdermal patch delivering a combination of B₁₂ and folate, or B₁₂, folate and B₆, in concentrations sufficient to reduce elevated homocysteine levels by treating either single or multiple deficiencies of B₁₂, folate, and B₆. The Allen Non-prescription formulations include 0.3-10 mg CN-cobalamin (B₁₂) and 0.1-0.4 mg folate or 0.3-10 mg B₁₂, 0.1-0.4 mg folate, and 5-75 mg B₆. The Allen prescription formulations comprise between 0.3-10 mg CN-cobalamin (B₁₂) and 0.4-10.0 mg folate or 0.3-10 mg B₁₂, 0.4-1.0 mg folate, and 5-75 mg B₆.

Piperine, a component of the spice black pepper, increases the bioavailability of curcumin and epigallocatechin-3-gallate. Piperine also exhibits significant antioxidant activity of its own, as well as significant chemopreventative and immunomodulary effects. A daily dosage can contain, for example, at least about 2.5 mg of piperine, or from about 1 to about 100 mg, or from about 10 to about 20 mg of piperine. A preferred source is piper longum derived from black pepper and standardized as 90%+piperine.

It is preferred that these compositions be delivered orally and the components be prepared for ingestion in a manner that makes the composition available in therapeutically effective amounts. As such, they may be prepared as water soluble compositions, delivered in liquid form, lyophilized, encapsulated, or in a manner suitable for time release, delayed release or enteric delivery, or any manner typically used for orally delivered pharmaceuticals, nutraceuticals or vitamins, or combined with foods or other normally ingested products. However, the invention is not limited to oral delivery as the compositions set forth herein may also be delivered by nasal spray, inhalation techniques, transdermally, transmucossal, by suppository, injected or by intravenous methods, including, for example, cognitive effects and biological markers associated with AD.

Although the above components are shown to have some effect on cognitive development, there is no prior evidence that these materials, alone or in combination, can delay the onset, arrest or reverse the development of, or treat behavioral or physiological effects of AD. In fact, to date, there is no effective treatment for AD. It has surprisingly been found that a combination of these ingredients dramatically treats AD.

In some embodiments, the compositions of the present invention comprise curcumin, piperine, epigallocatechin-3-gallate and N-acetylcysteine. The composition can comprise, for example, at least about 75 mg curcumin, at least about 0.6 mg piperine, at least about 35 mg epigallocatechin-3-gallage and at least about 32 mg N-acetylcysteine. The compositions can further comprise one or more of α-lipoic acid, vitamin B₁, vitamin B₆, vitamin B₁₂, folate, vitamin C and/or vitamin E. Compositions according to the present invention are therapeutically effective to treat a cognitive or neurological disorder, such as, for example, AD, in a patient.

In some embodiments, a composition according to the present invention comprises 500 mg of NAC, 100 mg of EGCG from green tea extract, 300 mg of alpha lipoic acid, 5 mg of folic acid, 1,000 mcg of hydroxycobalamin (vitamin B₁₂, 50 mg of pyridoxal-5-phosphate and/or 50 mg of pyridoxamine (B₆), 1,000 mg of turmeric (95% curcumin), 25 mg of vitamin B₁ (benfotiamine), 300 mg of vitamin C and optionally 400 IU of tocopheryl succinate (vitamin E) and/or 25 mg of vitamin B₂. An example composition, in which concentrations are expressed in percent gross weight, is listed in Table 3.

TABLE 3
Preferred Medical Food Cocktail

| | % Gross |
|---|---|
| Cocktail Ingredient | Weight |
| Turmeric (standardized to 95% curcuminoids) | 32.1% |
| Piper longum (black pepper standardized to 95% + | 0.7% |
| piperine) | |
| Green tea extract (standardized to 50% | 6.4% |
| Epigallocatechin-3-gallate | |
| R-α-Lipoic Acid | 9.6% |
| N-Acetylcysteine | 16.0% |
| B₁ (Benfotiamine/Thiamine pyrophosphate) | 2.7% |
| B₆ (Pyroxidal-5-phosphate/pyridoxamine) | 5.3% |
| B₁₂ (Hydroxycobalamin) | 3.2% |
| Folic Acid/Folate | 1.6% |
| Vitamin C (Ascorbic acid/Dehydroascorbic acid) | 9.6% |
| Vitamin E (Tocopherol succinate) | 12.8% |

The percent gross weight for each ingredient in the cocktail was determined by scaling up the elemental or therapeutic levels for each ingredient by its total weight as provided by the raw material suppliers. For example, if the anticipated therapeutic level of EGCG is 100 mg and the green tea extract used is 50% EGCG, then the gross weight of the green tea extract would be 200 mg.

Standardization of the content of all herbal products (tumeric, piper longum, and green tea) was confirmed by certificate of analysis from the supplier and also by assay by an independent laboratory of the herbal products.

Turmeric and green tea were obtained from USA NutraSource (City of Industry Calif.), black pepper was obtained from Sabinsa Corporation (Piscataway, N.J.), benfotiamine (B₁), pyridoxamine (B₆), and hydroxycobalamin (B₁₂) were obtained from Sigmna Aldrich Corporation (St. Louis, Mo.), N-acetylcysteine was obtained from Ashland Chemical (Cleveland, Ohio), a-Lipoic acid, vitamin B₁₂, folic acid, and vitamin E were obtained from Stauber Ingredients (Fullerton, Calif.) and vitamin C in the form of ascorbic acid and dehydroascorbic acid was obtained from Harmony Concepts (Eugene, Oreg.).

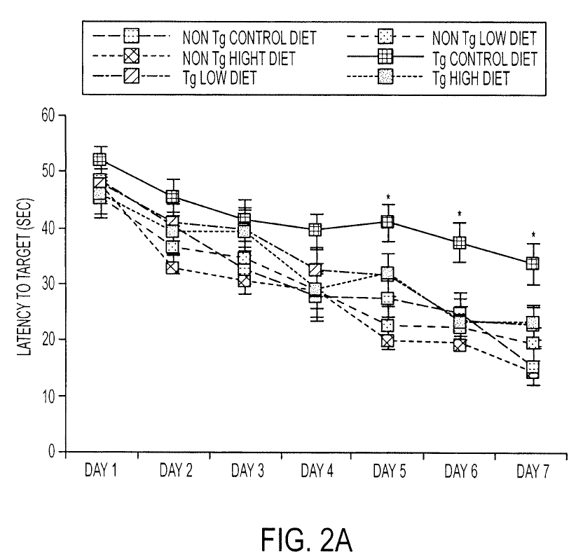

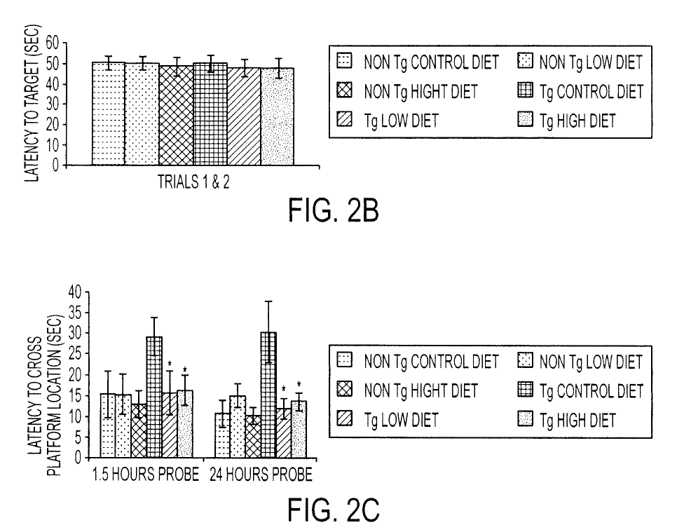

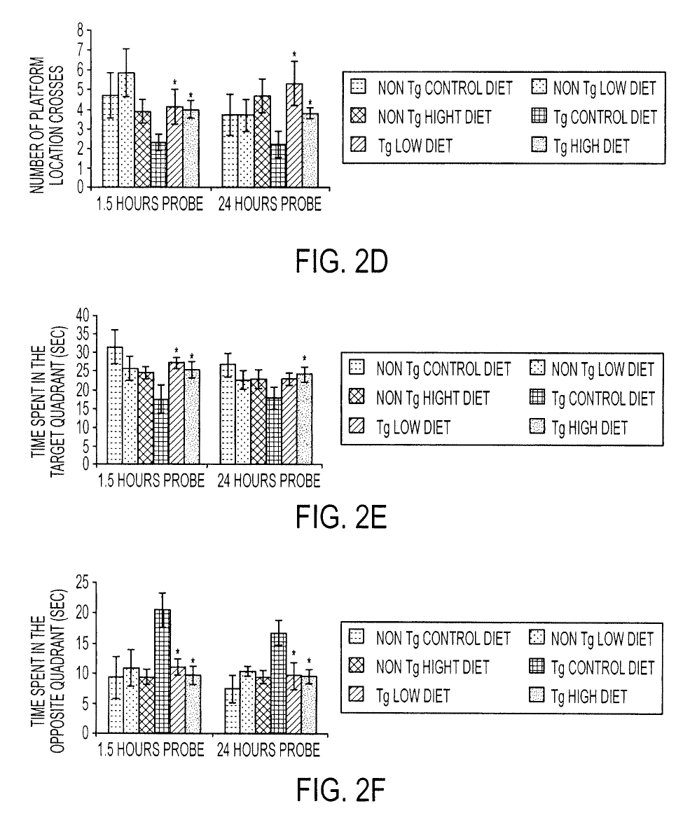

The compositions according to the present invention have been shown to prevent deterioration in cognitive performance on hippocampal and cortical dependent tasks in two different mouse models of Alzheimer's disease. Transgenic mice that are predisposed to develop Abeta plaques performed markedly worse than non-transgenic mice on a series of well-regarded cognitive tasks. However, transgenic mice that are administered the inventive compositions were statistically indistinguishable from non-transgenic mice in their performance on the same cognitive tasks (see, e.g., FIGS. 2A-F, 3, 5A). These results provide powerful evidence that the inventive compositions are effective in treating the cognitive deficits that arise in AD.

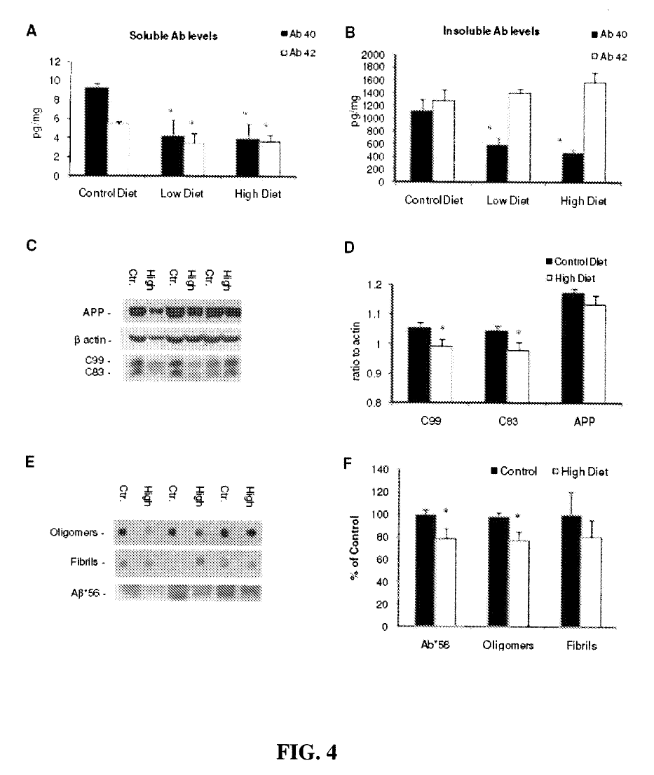

Furthermore, the inventive compositions have been shown to have beneficial effects on the levels of physiological markers of AD. For example, administration of the inventive compositions in two well-regarded mouse models of AD has been shown to result in marked decreases in the levels of, for example, soluble Aβ42, C99, and Aβ*56, all of which have been demonstrated to contribute to the physiological progression of AD (see, e.g., FIGS. 4A-F, 5D). These results demonstrate that the inventive compositions are effective in reducing the levels of the physiological markers of AD, a vital step in slowing or reversing the progression of AD.

Accordingly, the inventive compositions provide a surprising an unexpected advance in the art, as no effective treatment for AD existed prior to the present invention. Furthermore, the inventive compositions are especially beneficial because they include all-natural ingredients that are generally well-tolerated in patients.

### EXAMPLES

### Example 1

### Therapeutic Evaluation in Tg2576 Mice

### Example 1a: Cognitive Evaluation

We theorized that a combination therapy comprising a variety of antioxidants and vitamins would prove most efficacious in the treatment of AD in humans. To support this we have tested the following combination of nutraceuticals in two well-regarded and utilized mouse models of AD:

TABLE 4
Components of cocktail concentrate as well as low and high
concentration diets added to AIN-17 rodent chow

| | Cocktail | | Medium | | Low | |
|---|---|---|---|---|---|---|
| | Concentrate | | Concentration | | Concentration | |
| | (mg) | | Diet (mg) | | Diet (mg) | |
| Curcumin | 111 | mg | 33.3 | mg | 11.1 | mg |
| Piperine | 0.9 | mg | 0.27 | mg | .09 | mg |
| EGCG | 55 | mg | 16.5 | mg | 5.5 | mg |
| a-Lipoic Acid | 28 | mg | 8.4 | mg | 2.8 | mg |
| N-Acetylcysteine | 47 | mg | 14.1 | mg | 4.7 | mg |
| Vitamin B₁ | 4.7 | mg | 1.41 | mg | .47 | mg |
| Vitamin B₆ | 9.4 | mg | 2.82 | mg | .94 | mg |
| Vitamin B₁₂ | .022 | mg | 0.0066 | mg | .0022 | mg |
| Folate | .06 | mg | 0.018 | mg | .006 | mg |
| Vitamin C | 36 | mg | 10.8 | mg | 3.6 | mg |
| Total | 292.1 | mg | 87.63 | mg | 29.21 | mg |

The high concentration diet and low concentration diet cocktails were prepared to provide the above amounts of components per 1 kg rodent chow. The mice each ate about 4-5 g rodent chow each day. The mice ranged in weight from about 20-25 g each. Accordingly, mice on the low concentration diet consumed at least about 4.6 mg/kg/day of the cocktail, and mice on the high concentration diet may have eaten as much as 73 mg/kg/day of the cocktail.

The diets were fed to Tg2576 and non-trangenic (nonTg) control mice at 6 months of age. After 6 months of treatment mice were tested for cognition on hippocampal and cortical dependent tasks. The first task used was the widely utilized Morris water maze (FIG. 2A-F). This task tests spatial memory and consists of 4 daily trials over 7 days, in which mice must learn the location of a hidden platform. As mice acquire the task they should reach the platform quicker, indicating improved spatial memory. Acquisition curves demonstrated that 12-month-old Tg2576 mice were severely cognitively impaired compared to age-matched nonTg mice. (FIG. 2A.) However, Tg2576 mice treated with either the low or high concentration diet acquired the task significantly better than untreated Tg2576 mice, and were statistically indistinguishable from nonTg mice. These results show that the cocktail-containing diets prevent cognitive deficits associated with development of AD pathology on hippocampal spatial acquisition. NonTg mice treated with the cocktail-containing diet acquired the task to a similar degree as the untreated nonTg mice. In order to ensure that all mice were starting off at the same level we averaged the first two trials of the first day of training for each group (FIG. 2B). All groups were statistically insignificant from one another, showing that all groups initially performed equally, but then learned the task at different rates. Spatial reference memory probe trials were conducted at 1.5-h and 24-h after the last training trial to examine short and long-term memory, respectively. Consistent with the acquisition curves, Tg2576 mice showed impaired latencies to cross the platform location as compared to nonTg mice at both the 1.5 and 24 h probes (FIG. 2C). Tg2576 mice treated with cocktail-containing diet performed at untreated nonTg levels, thus completely preventing the deficits seen in the untreated Tg2576 mice. Similar results were seen in the number of platform crosses (FIG. 2D), time spent in the target quadrant (FIG. 2E), and time spent in the opposite quadrant (FIG. 2F). These results show complete prevention of cognitive deficits seen in Tg2576 mice, which arise due to AD-like pathology, by treatment with either a low or high concentration diet.

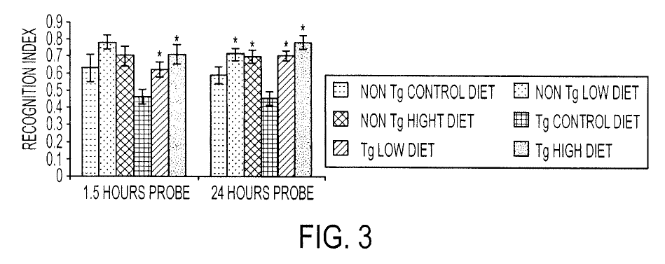

Finally, we evaluated cocktail diet-treated and untreated Tg2576 and nonTg mice in performance of the cortex-dependent contextual task, novel object recognition, which relies on the animals' preference to explore a novel object over a familiar object. After familiarization with the object, mice were reintroduced to the familiar, as well as a novel, object 1.5 and 24 h later. Their ability to remember which object they had seen before was then assessed. Tg2576 mice explored at chance level, indicating that they were not discriminating between the 2 objects suggesting that they could not recall the familiar object. NonTg mice spent significantly more time with the novel object, showing that they could recall the familial object (FIG. 3). Treatment with cocktail-containing diet significantly improved nonTg performance at the 24-hour probe, suggesting that treated nonTg mice had improved cortical dependent memory compared to untreated nonTg mice. Notably, treatment of Tg2576 fully restored performance to nonTg levels at both 1.5- and 24-hour probe trials. These results show that either low or high concentration diet fully prevented cortical cognitive deficits, which arise due to AD-like pathology in the Tg2576 mice.

### Example 1b—Physiological Evaluation

To assess the disease modifying effects of the cocktail-containing diet, we looked at brain pathology in untreated Tg2576 mice and high and low concentration diets treated Tg2576 mice. Levels of soluble Aβ40 and 42 were significantly reduced for both treatment groups (FIG. 4A); levels of insoluble Aβ40 were also significantly reduced (FIG. 4B). Soluble Aβ42 is the form most often associated with disease states including AD. Given these striking reductions in Aβ levels we assessed levels of its precursors C99 and APP. APP is cleaved by a-secretase to form C83 and an n-terminal fragment, whereas β-secretase cleaves APP into C99 and an n-terminal fragment. C99 and C83 are both cleaved by gamma-secretase. Cleavage of C99 by gamma-secretase results in abeta.

We found no changes in steady state levels of APP but significant decreases in C99, and also C83 (FIG. 4C, D). These results show that the cocktail-containing diet is disease modifying, exerting a direct effect on physiological markers for AD, in addition to being able to prevent cognitive deficits in Tg2576 mice.

A tremendous amount of recent evidence has highlighted the aggregation state of Aβ in being important to its pathological activities, rather than just its levels. Low molecular weight oligomeric Aβ species have been highlighted as the most toxic, and have been shown to be potent inhibitors of long-term potentiation (LTP), a form of synaptic plasticity thought to underlie memory, as well as proteasome function leading to the accumulation of other intracellular proteinaceous aggregates. As such, therapies that target the breakdown of these oligomers are of great interest. To assess levels of low molecular weight oligomeric Aβ species we used the conformation specific antibody A11. Dot blot analysis showed a 50% reduction of these toxic soluble oligomers in the brains of Tg2576 animals treated with the high concentration diet (FIG. 4E). A soluble Aβ dodecamer designated Aβ*56 has been highlighted as an oligomeric species that causes memory deterioration and synaptic dysfunction. Analysis of this Aβ dodecamer revealed a 50% reduction in Tg2576 mice treated with the high concentration diet, compared to untreated mice (FIG. 4F). Such a therapy is of enormous potential, and is the only oral treatment to date shown to affect this crucial Aβ species.

### Example 2: Therapeutic Evaluation in 3×Tg-AD Mice

Given these extremely promising results we sought to validate the cocktail-containing diet by testing the high-concentration diet in a second mouse model of AD, the 3×Tg-AD mouse model. The 3×Tg-AD mice progressively develop AB and tau pathology, with a temporal- and regional-specific profile that closely mimics their development in the human AD brain. Despite equivalent expression of the human APP and human tau transgenes, Aβ deposition develops prior to the tangle pathology. Extracellular Aβ deposits manifest by 6 months of age in the cortex, and by 12 months are thioflavin S-positive and also positive for Congo red. Tau becomes mislocalized to the somatodendritic compartment at ˜6 months of age, and reactivity with conformational specific antibodies such as the mouse monoclonal antibodiy MC-1 is apparent by 10 months, followed shortly thereafter by immunoreactivity for phospho-specific tau markers at about 10-12 months of age. The tau pathology follows a hierarchal pattern, with MCI immunoreactivity emerging first, followed by phospho-specific markers such as AT8 and AT180, and then lastly PHF. This closely mimics the pathology that occurs in the human brain.

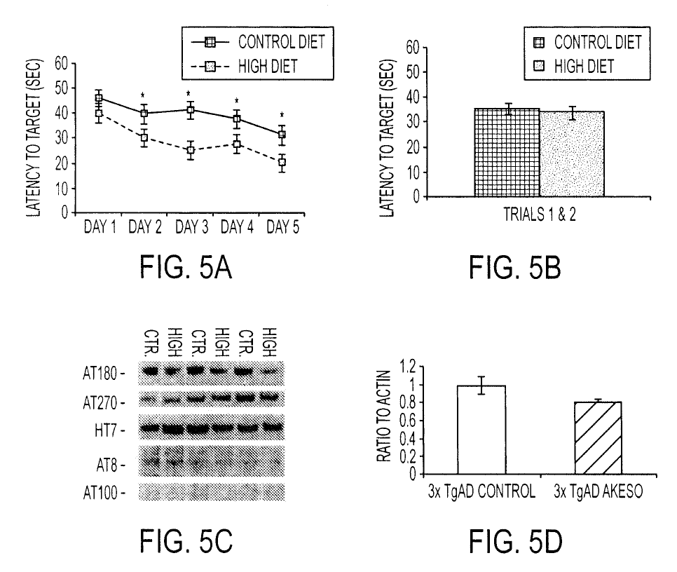

3×Tg-AD mice were 4 months of age at the beginning of treatment. Hippocampal dependent spatial memory was assessed five months later using the Morris water maze. In accordance with the Tg2576 mice we found that the combination diet treated 3×Tg-AD mice performed significantly better than the untreated 3×Tg-AD mice on every day of training except the first (FIG. 5A-B). This indicates that the combination diet treated mice learn the task at a faster rate than the untreated mice. In addition, administration of the high concentration diet affected numerous physiological markers for AD (FIG. 5C-D). For example, phosphorylation of tau protein at threonine 231 was significantly reduced, as measured by AT180 levels (FIG. 5D).

Taken together, these results show that our nutraceutical combination diet is effective at preventing cognitive decline associated with AD pathology in 2 different mouse models of AD. Furthermore, the diet has disease-modifying properties. This combination diet thus represents a highly promising treatment for human AD and meets an urgent need in the art for AD therapies, especially ones, such as this one, that are likely to be extremely safe and well tolerated in humans.

The embodiments illustrated and discussed in this specification are intended only to teach those skilled in the art the best way known to the inventors to make and use the invention. Nothing in this specification should be considered as limiting the scope of the present invention. All examples presented are representative and non-limiting. The above-described embodiments of the invention may be modified or varied, without departing from the invention, as appreciated by those skilled in the art in light of the above teachings. It is therefore to be understood that, within the scope of the claims and their equivalents, the invention may be practiced otherwise than as specifically described.

What is claimed is:

1. A composition comprising curcumin, piperine, epigallocatechin-3-gallate and N-acetylcysteine in such amounts that the composition is therapeutically effective to treat a cognitive or neurological disorder in a patient.

2. The composition of claim 1, wherein the cognitive or neurological disorder is Alzheimer's disease.

3. The composition of claim 1, wherein the cognitive or neurological disorder is amyotrophic lateral sclerosis, mild cognitive impairment, frontotemporal dementia with Parkinsonism linked to chromosome 17, Pick's disease, progressive supranuclear palsy, and corticobasal degeneration.

4. The composition of claim 1, comprising at least about 75 mg curcumin, at least about 0.6 mg piperine, at least about 35 mg epigallocatechin-3-gallate, and at least about 32 mg N-acetylcysteine.

5. The composition of claim 4, further comprising one or more ingredients selected from the group consisting of:
α-lipoic acid in an amount of at least about 19 mg;
vitamin B₁ in an amount of at least about 3 mg;
vitamin B₆ in an amount of at least about 6 mg;
vitamin B₁₂ in an amount of at least about 0.02 mg;
folate in an amount of at least about 0.04 mg;
vitamin C in an amount of at least about 24 mg;
and combinations thereof.

6. The composition of claim 4, further comprising:
α-lipoic acid in an amount of at least about 19 mg;
vitamin B₁ in an amount of at least about 3 mg;
vitamin B₆ in an amount of at least about 6 mg;
vitamin B₁₂ in an amount of at least about 0.02 mg;
folate in an amount of at least about 0.04 mg; and
vitamin C in an amount of at least about 24 mg.

7. A method of treating a cognitive or neurological disorder in a patient, comprising administering to the patient a therapeutically effective amount of a composition comprising curcumin, piperine, epigallocatechin-3-gallate and N-acetylcysteine.

8. The method of claim 7, wherein the cognitive or neurological disorder is Alzheimer's disease.

9. The method of claim 7, wherein the cognitive or neurological disorder is amyotrophic lateral sclerosis, mild cognitive impairment, frontotemporal dementia with Parkinsonism linked to chromosome 17, Pick's disease, progressive supranuclear palsy, and corticobasal degeneration.

10. The method of claim 7, wherein the composition is administered in an amount sufficient to provide at least about 1.05 mg/kg patient body weight curcumin, at least about 0.01 mg/kg patient body weight piperine, at least about 0.5 mg/kg patient body weight epigallocatechin-3-gallate, and at least about 0.4 mg/kg patient body weight N-acetylcysteine.

11. The method of claim 10, wherein the composition is administered in unit dosage form comprising one unit dosage daily.

12. The method of claim 10, wherein the composition is administered in unit dosage form comprising more than one unit dosage daily.

13. The method of claim 10, wherein the composition is administered in an amount sufficient to provide from about 1.05 to about 85 mg/kg patient body weight curcumin, from about 0.01 to about 1.0 mg/kg patient body weight piperine, from about 0.5 to about 40 mg/kg patient body weight epigallocatechin-3-gallate, and about 0.4 to about 35 mg/kg patient body weight N-acetylcysteine.

14. The method of claim 10, wherein the composition is administered in an amount sufficient to provide from about 11.1 to about 111 mg/kg patient body weight curcumin, from about 0.09 to about 0.9 mg/kg patient body weight piperine, from about 5.5 to about 55 mg/kg patient body weight epigallocatechin-3-gallate, and about 4.7 to about 47 mg/kg patient body weight N-acetylcysteine.

15. The method of claim 10, wherein the composition is administered in an amount sufficient to provide from about 8.8 to about to about 13.4 mg/kg patient body weight curcumin, from about 0.09 to about 0.11 mg/kg patient body weight piperine, from about 4.4 to about 6.6 mg/kg patient body weight epigallocatechin-3-gallate, and about 3.7 to about 5.7 mg/kg patient body weight N-acetylcysteine.

16. The method of claim 10, wherein the composition is administered in an amount sufficient to provide from about 88.8 to about 133.2 mg/kg patient body weight curcumin, from about 0.7 to about 1.1 mg/kg patient body weight piperine, from about 44 to about 66 mg/kg patient body weight epigallocatechin-3-gallate, and about 37 to about 56.4 mg/kg patient body weight N-acetylcysteine.

17. The method of claim 10, wherein the composition further comprises one or more ingredients selected from the group consisting of α-lipoic acid, vitamin B₁, vitamin B₆, vitamin B₁₂, folate, and vitamin C and is administered in an amount sufficient to provide as least one of:
α-lipoic acid in an amount of at least about 0.2 mg/kg patient body weight;
vitamin B₁ in an amount of at least about 0.05 mg/kg patient body weight;
vitamin B₆ in an amount of at least about 0.09 mg/kg patient body weight;
vitamin B₁₂ in an amount of at least about 0.0002 mg/kg patient body weight;
folate in an amount of at least about 0.0006 mg/kg patient body weight; or
vitamin C in an amount of at least about 0.35 mg/kg patient body weight.

18. The method of claim 10, wherein the composition further comprises α-lipoic acid, vitamin B₁, vitamin B₆, vitamin B₁₂, folate, and vitamin C and is administered in an amount sufficient to provide:
α-lipoic acid in an amount of at least about 0.2 mg/kg patient body weight;
vitamin B₁ in an amount of at least about 0.05 mg/kg patient body weight;
vitamin B₆ in an amount of at least about 0.09 mg/kg patient body weight;
vitamin B₁₂ in an amount of at least about 0.0002 mg/kg patient body weight;
folate in an amount of at least about 0.0006 mg/kg patient body weight; and
vitamin C in an amount of at least about 0.35 mg/kg patient body weight.

19. The method of claim 18, wherein the cognitive or neurological disorder is Alzheimer's disease.

20. The method of claim 18, wherein the cognitive or neurological disorder is amyotrophic lateral sclerosis, mild cognitive impairment, frontotemporal dementia with Parkinsonism linked to chromosome 17, Pick's disease, progressive supranuclear palsy, and corticobasal degeneration.

21. The method of claim 7, wherein treating the cognitive or neurological disorder comprises treating one or more adverse cognitive symptoms associated with the cognitive or neurological disorder.

22. The method of claim 21, wherein the cognitive symptom is selected from the group consisting of memory loss, personality change, agitation, disorientation, loss of coordination, inability to care for one's self, and combinations thereof.

23. The method of claim 7, wherein treating the cognitive or neurological disorder comprises treating one or more adverse physiological symptoms associated with the cognitive or neurological disorder.

24. The method of claim 23, wherein the physiological symptom is selected from the group consisting of amyloid plaques, tau protein tangles, tau protein phosphorylation, microtubule destabilization, synaptic loss, and combinations thereof.

25. The method of claim 7, wherein treating the cognitive or neurological disorder comprises reducing a level of a low molecular weight oligomeric beta amyloid peptide in the patient.

26. The method of claim 25, wherein the oligomeric beta amyloid peptide is Aβ*56.

27. The method of claim 25, wherein treating the cognitive or neurological disorder comprises reducing levels of low molecular weight oligomeric amyloid beta peptide by at least about 50% in the patient.
